(19) United States
(12) Patent Application Publication
(10) Pub. No.: US 2025/0258182 A1
(43) Pub. Date: Aug. 14, 2025

(54) ROCKY MOUNTAIN SPOTTED FEVER DETECTION AND TREATMENT

(71) Applicant: IDEXX Laboratories, Inc., Westbrook, ME (US)

(72) Inventors: Joshua Michael Boucher, Gray, ME (US); Anton Mestek, Gorham, ME (US); Clever Madrid, Cumberland, ME (US); Jesse Stephen Buch, Kennebunk, ME (US)

(73) Assignee: IDEXX Laboratories, Inc., Westbrook, ME (US)

(21) Appl. No.: 19/041,631

(22) Filed: Jan. 30, 2025

Related U.S. Application Data
(63) Continuation of application No. 17/554,557, filed on Dec. 17, 2021, now Pat. No. 12,228,578.
(60) Provisional application No. 63/126,756, filed on Dec. 17, 2020.

(51) Int. Cl. G01N 33/68; C07K 14/29; G01N 33/569

(52) U.S. Cl. CPC G01N 33/6854; C07K 14/29; G01N 33/56911; C07K 2319/02; C07K 2319/30; G01N 2333/29; G01N 2469/20

(57) ABSTRACT

Provided herein are compositions and methods for detection and treatment of Rocky Mountain spotted fever (“RMSF”). The compositions specifically detect Rickettsia rickettsii and do not cross-react with other Rickettsia species providing rapid and accurate detection and diagnosis of RMSF.

### PRIORITY

This application is a continuation of U.S. Ser. No. 17/554,557, filed on Dec. 17, 2021, which claims the benefit of U.S. Ser. No. 63/126,756, filed on Dec. 17, 2020, which are incorporated by reference in their entireties.

### SEQUENCE LISTING

The instant application contains a Sequence Listing which has been submitted electronically, and is hereby incorporated by reference in its entirety. Said XML copy, created on Mar. 18, 2025, is named “761300_IDX-005CON_SL.xml” and is 31,813 bytes in size.

### BACKGROUND

Rocky Mountain Spotted Fever (RMSF) is a vector borne disease that is caused by Rickettsia rickettsii infection. It is challenging to identify the disease in dogs due to the cross-reacting antibodies from non-pathogenic Rickettsia spp. Therefore, identification of R. rickettsii specific genes/proteins is critical for the development of an effective diagnostic test. RMSF is a zoonotic disease that is transmitted to dogs mostly by American dog tick (Dermacentor variabilis), Rocky Mountain wood tick (Dermacentor andersonii) and other ticks such as Ambylomma and Rhipicephalus. Ticks infected with R. rickettsii bite humans, dogs, deer, and other animals thereby spreading the infectious bacteria. R. rickettsii enter the bloodstream during tick bite and invade the vascular cells of the microcirculatory system, often resulting in widespread vascular damage.

R. rickettsii is phylogenetically closely related to other Rickettsia spp and endosymbionts. Although the genomes of several different species of Rickettsia have been sequenced, the challenge for the identification of diagnostic antigen is the cross-reacting antibodies from both non-pathogenic or endosymbionts and other Rickettisia spp. The current diagnosis of RMSF is performed by PCR, IFA, and culture. However, these diagnostic methods have certain limitations in detecting the infection (Parola et al., (2005) Veterinary research, 36 (3), 469-492; Breitschwerdt et al., (1999) Antimicrobial agents and chemotherapy, 43 (4), 813-821; Levin et al. (2014) PloS one, 9 (12).

RMSF is one of the most lethal tick-borne diseases. Infection of humans with R. rickettsii can result in a ˜15% mortality rate if untreated. Human cases have been steadily on the rise over the last decade. The prevalence in canines can vary from as low as 0.1% in non-endemic areas to as high as 14% or greater in endemic areas or during a local outbreak. Novel markers are needed for R. rickettsii infection that will lead to early (days 4-14 DPI infection or earlier) and highly specific detection of anti-Rickettsia rickettsii antibodies. Furthermore, there is a need for a species-specific diagnostic test for R. rickettsii.

### SUMMARY

An embodiment provides a polypeptide comprising (i) 90% or more sequence identity to a polypeptide as set forth in SEQ ID NO: 1, 2, 3, 4, 5, 6, or 7; (ii) a fusion protein made up of two, three, four, five, six, seven or more polypeptides having 90% or more sequence identity to SEQ ID NO:1, 2, 3, 4, 5, 6, or 7; or (iii) a fusion protein made up of at least two polypeptides having 90% or more sequence identity to SEQ ID NO:1, 2, 3, 4, 5, 6, or 7.

Another embodiment provides a polypeptide having less than 75 total amino acids and comprising 90% or more sequence identity to a polypeptide as set forth in SEQ ID NO:1, 2, 5, 6, or 7.

Yet another embodiment provides a polypeptide having less than 350 total amino acids and comprising 90% or more sequence identity to a polypeptide as set forth in SEQ ID NO:3 or 4.

In an embodiment the polypeptides are not naturally occurring. The polypeptides can be lyophilized, desiccated, or dried. The polypeptides can further comprise one or more labels or tags. The polypeptides can be immobilized to a support. The polypeptides can be present in an immunocomplex with one or more antibodies that specifically bind to a polypeptide as set forth in SEQ ID NO:1, 2, 3, 4, 5, 6, or 7.

Still another embodiment provides a method of detecting anti-Rickettsia rickettsii antibodies or specific binding fragments thereof. The method can comprise contacting a test sample with one or more polypeptides comprising 90% or more sequence identity to a polypeptide as set forth in SEQ ID NO:1, 2, 3, 4, 5, 6, or 7; and detecting complexes of anti-Rickettsia rickettsii antibodies or specific binding fragments thereof and the one or more polypeptides. The one or more polypeptides can be immobilized to a support. The complexes can be detected using one or more secondary antibodies or specific binding fragments thereof that specifically bind anti-Rickettsia rickettsii antibodies or specific binding fragments thereof. The secondary antibodies or specific binding fragments thereof can comprise one or more tags or labels. The complexes can be detected using one or more detector polypeptides comprising 90% or more sequence identity to a polypeptide as set forth in SEQ ID NO: 1, 2, 3, 4, 5, 6, or 7. The one or more detector polypeptides can comprise a label or tag. The polypeptide can comprise 90% or more sequence identity to a polypeptide as set forth in SEQ ID NO: 1, 2, 5, 6, or 7 and further can include one or more secretory signal sequences, one or more epitope tags, or one or more secretory signal sequences and one or more epitope tags.

Another embodiment provides a method for diagnosing a disease caused by Rickettsia rickettsii in a subject. The method can comprise contacting a test sample with one or more polypeptides comprising 90% or more sequence identity to a polypeptide as set forth in SEQ ID NO:1, 2, 3, 4, 5, 6, or 7; and detecting complexes of anti-Rickettsia rickettsii antibodies or specific binding fragments thereof and the one or more polypeptides. The subject can be infected with Rickettsia rickettssi for less than about 4, 5, 6, 7, 8, or 9 days. The method can further comprise comparing an amount of the complexes in the sample to a control sample or control standard, wherein elevated levels of the complexes as compared to the control sample or control standard is an indication of a disease caused by Rickettsia rickettsii. The method can further comprise administering a treatment for a disease caused by Rickettsia rickettsii where the complexes are detected. The method can further comprise determining an amount of the anti-Rickettsia rickettsii antibodies or specific binding fragments thereof in the sample. The subject can be a human or a non-human animal. The test sample can be whole blood, plasma, serum, lymph fluid, urine, feces, nasal swab, throat swab, saliva, an environmental sample, or any other suitable sample.

In an embodiment, the anti-Rickettsia rickettsii antibodies or specific binding fragments thereof can be detected by a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, a western blot assay, surface plasmon resonance (SPR), isothermal titration calorimetry (ITC), microscale thermophoresis (MST), biolayer interferometry, or grating-coupled interferometry.

Yet another embodiment provides a kit for diagnosing a disease caused by Rickettsia rickettsii. A kit can comprise one or more polypeptides comprising 90% or more sequence identity to a polypeptide as set forth in SEQ ID NO: 1, 2, 3, 4, 5, 6, or 7, wherein the polypeptide is not naturally occurring; and one or more reagents that facilitate binding of the one or more polypeptides to anti-Rickettsia rickettsii antibodies or specific binding fragments thereof present in a test sample.

### BRIEF DESCRIPTION OF THE DRAWINGS

FIG. 1 shows a hypothetical time course for RMSF infection and available diagnostics at selected times post infection. Severity, mortality risk, and seroconversion are dependent on the dose of pathogen, immune response of the infected and strain of Rickettsia. The x-axis represents days post infection (DPI) and the y-axis represents severity of infection. The curve is a representative time-course of RMSF infection. As infection progresses over time, the afflicted will either seroconvert and eliminate the infection or succumb to multi-system failure and potentially death. The insert shows a longitudinal section of R. rickettsii infected vasculature. The arrow with doxycycline represents the time window for effective treatment. R. rickettsii are evolutionarily primed to rapidly exit the blood stream and replicate in the vascular endothelial cells (see insert), thus rendering PCR from a blood draw ineffective beyond the first few days of infection. IFA which detects seroconversion lacks specificity and is generally very laborious, rendering it ineffective for rapid or in-clinic guidance on treatment.

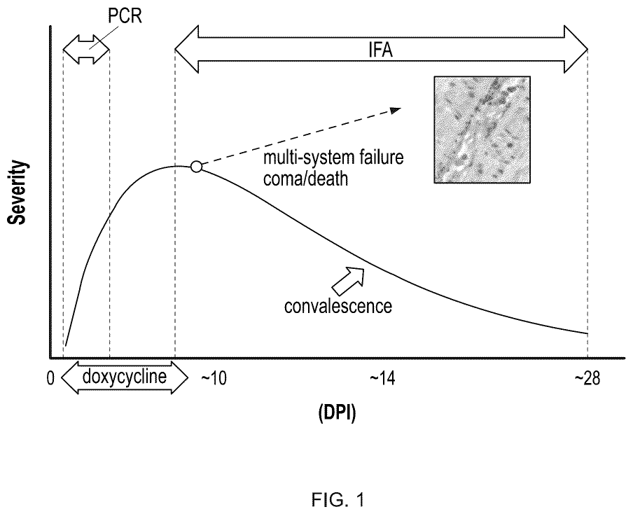

FIG. 2 shows a schematic of the extracellular protein SCA2 in the top pane. In the autochaperone domain (AC) of the extracellular protein SCA2, boxed, resides a stretch of amino acids that is only present in R. rickettsii strains. The bottom pane shows sequences for the SCA2 auto chaperone region from Rickettsia species. The entire unique region (larger horizontal box) is PDX6, while a slightly truncated region (smaller horizontal box) is PDX7.

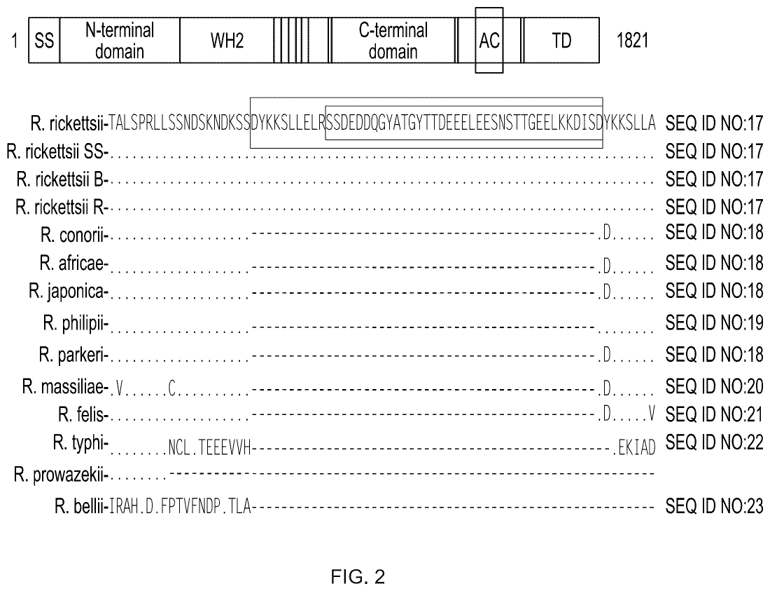

FIG. 3 shows specific reagents generated around the R. rickettsii-specific region of Sca2 protein. PDX6 is representative of the whole unique region of interest, while PDX7 is a slightly truncated sequence. Two recombinant fusion proteins were made consisting of 3 repeats of the PDX6 region (TDX1779) or 5 repeats of the PDX6 region (TDX1780). Recombinant proteins were also synthesized with 8× His tags and epitope tags.

FIG. 4 shows a schematic of the PDX7 polypeptide conjugated to amino beads with a primary and secondary antibody. Detection of antibody binding was reported using biotinylated rabbit anti canine IgG.

FIG. 5 shows PDX7 reactivity to experimental (pooled) sera on multiplex. PDX7 fluorescence intensity (MFI) of PDX7 before infection and 4, 7, 11, 14, 21, 25, 32, and 39 days post infection (PDI). PDX7 was conjugated to amino beads and incubated against pooled serum from R. rickettsii experimentally infected canines. Detection of the antibody binding was reported using biotinylated rabbit anti-canine IgG. The Ideal treatment window lies in the range between PID4 and PID21.

FIG. 6 shows TDX1779 and 1780 reactivity to experimental sera on multiplex (Indirect anti-species format). TDX1779 and TDX1780 were coupled to carboxy beads and incubated against serum from R. rickettsii experimentally infected canines (Dog 1 or Dog 6) (Post infection day=PID). Detection of the antibody binding was reported using biotinylated Rabbit anti canine IgG.

FIG. 7 shows a schematic of the direct assay format in which anti-R. rickettsii antibodies are captured by PDX7 conjugated to amino beads and detected by biotinylated PDX7 peptide instead of biotinylated anti-species antibody.

FIG. 8 shows a PDX7 peptide conjugated to amino beads and screened against 574 samples from the United States using a direct assay format in which biotinylated peptide is used as the detector in place of the anti-species IgG (direct format, see FIG. 7). Using this approach, 8 positive samples were identified out of 574 tested. The 8 positives were further evaluated using an RMSF Immunofluorescent assay (IFA) for confirmation of positivity. Of the 8 PDX7 positive samples identified by multiplex, six were confirmed positive by IFA, suggesting strong sensitivity and predictive value of PDX7 peptide for capturing anti-Rickettsia antibodies.

FIG. 9 shows reactivity of serum from R. rickettsii experimentally infected canines to PDX21 polypeptides.

### DETAILED DESCRIPTION

The shell vial technique is currently the most definitive diagnostic method for RMSF. This technique involves inoculating a clinical specimen onto a cell monolayer grown on a cover slip in a shell vial culture tube, low speed centrifugation to enhance viral infectivity, and then incubation. This technique, however, has serious drawbacks. Approximately one-third of the isolates can be lost when passaged to new cells. Furthermore, the culture technique is limited to laboratories equipped with biosafety level 3 facilities and staff capable of maintaining living host cells (animal mouse models or embryonated eggs), or cell cultures (Vero, L929, HEL, XTC-2, or MRC5 cells).

The standard serologic test for diagnosis of RMSF is an indirect immunofluorescence assay (IFA) for immunoglobulin G (lgG) using R. rickettsii pathogens fixed to slides. This assay, however, is not specific for R. rickettsii. It also detects, for example, R. akari, R. parkeri, and Rickettsia 364D. Cross-reactivity among antigens of pathogens within the same genus and occasionally in different genera is a known major limitation of serology. IFAs also suffer from non-specific signal as a result of antibody cross reactivity and/or anti-species antibody non-specifically binding the slides. As a further limitation, in areas where no Rickettsia pathogens have previously been isolated or detected, IFA assays cannot be relied upon as a sole method of diagnosis, but only as a cross reference technique.

Polymerase chain reaction (PCR) assays and sequencing methods can also be used to detect and identify RMSF in blood and skin biopsies. Though PCR and sequencing methods are rapid, traditional PCR has poor sensitivity for detecting R. rickettsii in blood samples and samples can be contaminated. A nested PCR assay, such as the proposed “suicide PCR” assay, attempts to mitigate vertical contaminations by amplicons from previous assays. The suicide PCR assay uses single-use primers targeting a gene never amplified previously in the laboratory. Nevertheless, standard nested PCR assays are highly subject to contamination and false-positive results.

A diagnostic method for RMSF that can specifically detect Rickettsia rickettsii and not cross-react with other Rickettsia species would contribute to rapid and accurate diagnosis of RMSF. A diagnostic test that offered such advantages while mitigating the drawbacks of culture, IFA, and PCR methods would ensure more accurate and timely diagnosis of RMSF in a larger portion of the global population. A faster and more accurate diagnostic test for RMSF would relay into earlier treatment, which is important as mortality rates for RMSF increase when treatment is delayed.

Provided herein are compositions and methods for detection and treatment of RMSF. The compositions can specifically detect Rickettsia rickettsii and do not likely cross-react with other Rickettsia species providing rapid and accurate diagnosis of RMSF.

### Polypeptides

A polypeptide is a polymer where amide bonds covalently link three or more amino acids. A polypeptide can be post-translationally modified. A purified polypeptide is a polypeptide preparation that is substantially free of cellular material, other types of poly peptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or less of other polypeptides, culture medium, chemical precursors, and/or other chemicals used in synthesis. Therefore, a purified polypeptide is about 70%, 80%, 90%, 95%, 99% or more pure.

The term “polypeptides” can refer to one or more types of polypeptide or a set of polypeptides. “Polypeptides” can also refer to mixtures of two or more different types of polypeptides including, but not limited to, full-length proteins, truncated polypeptides, or polypeptide fragments. The term “polypeptides” or “polypeptide” can each mean “one or more polypeptides.”

### SCA2 Polypeptides

The Rickettsia SCA2 protein is one of 17 surface cell antigen (SCA) proteins. Surface cell antigen (SCA) proteins refer to the family of Rickettsia autotransporters (ATs). These SCA proteins are involved in bacterial infection of the host cell. The SCA2 protein is highly conserved across Rickettsia species and is an outer membrane protein that can facilitate adhesion invasion and motility of Rickettsia with target cells. Examples of the polypeptide sequence of SCA2 is shown in GenBank Accession number AJG33947.1 and AJG32613.1. SCA2 functions, for example, as a formin mimic that is responsible for actin-based motility of Rickettsia in the host cell cytosol.

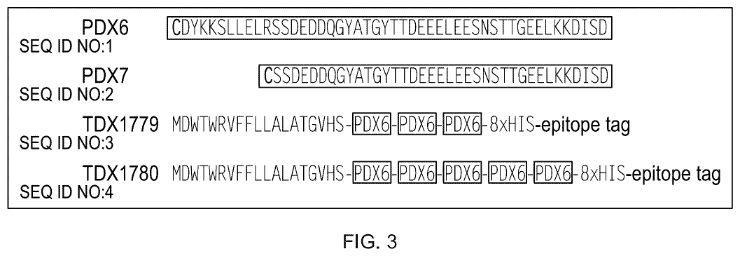

A stretch of amino acids that is only present in R. rickettsii strains exists in the autochaperone domain (AC) of the extracellular protein SCA2. The entire unique region called PDX6 herein, while a slightly truncated region is named PDX7 herein. See FIG. 3. Additionally, two recombinant fusion proteins have been constructed, TDX1779 and TDX1780, by fusion of 3 PDX6 repeats or 5 PDX6 repeats, respectively. Any of these polypeptides can be immobilized to a support such as, for example, barcoded magnetic beads (BMBs) and used in a detection assay.

(PDX6)
SEQ ID NO: 1
CDYKKSLLXLRSSDEDDQGYATGYTTDEEELEEXNSTTGEELKKDISD

The X at position 9 can be E or A. The X at position 34 can be G or S.

(PDX7)
SEQ ID NO: 2
CSSDEDDQGYATGYTTDEEELEEXNSTTGEELKKDISD

The X at position 24 can be G or S.

(TDX1779)
SEQ ID NO: 3
MDWTWRVFFLLALATGVHSCDYKKSLLELRSSDEDDQGYATGYTTDEE
ELEESNSTTGEELKKDISDDYKKSLLELRSSDEDDQGYATGYTTDEEE
LEESNSTTGEELKKDISDDYKKSLLELRSSDEDDQGYATGYTTDEEEL
EESNSTTGEELKKDISDAAAHHHHHHHH

The structure of TDX1779 is (MDWTWRVFFLLALATGVHS-PDX6-PDX6-PDX6-8XHIS) (see FIG. 3). The underlined portion of the sequence at the N terminus (e.g., MDWTWRVFFLLALATGVHS SEQ ID NO:24) is a secretory (signal) sequence. The “8XHIS” means a His tag comprising 8 His residues is present.

(TDX1780)
SEQ ID NO: 4
MDWTWRVFFLLALATGVHSCDYKKSLLELRSSDEDDQGYATGYTTDEE
ELEESNSTTGEELKKDISDDYKKSLLELRSSDEDDQGYATGYTTDEEE
LEESNSTTGEELKKDISDDYKKSLLELRSSDEDDQGYATGYTTDEEEL
EESNSTTGEELKKDISDDYKKSLLELRSSDEDDQGYATGYTTDEEELE
ESNSTTGEELKKDISDDYKKSLLELRSSDEDDQGYATGYTTDEEELEE
SNSTTGEELKKDISDAAAHHHHHHHH

The structure of TDX1780 is (MDWTWRVFFLLALATGVHS-PDX6-PDX6-PDX6-PDX6-PDX6-PDX6-8XHIS). See FIG. 3.

The underlined amino acids indicate non-natural variation and/or additions to the polypeptide sequence that were engineered for biochemical/immunological purposes. These polypeptides are therefore non-naturally occurring and have different properties than naturally occurring polypeptides including, for example, better purification properties and better specificity and sensitivity in assays.

A secretory signal sequence is a peptide sequence (or polynucleotide encoding the peptide sequence) present at the N-terminus (or in some cases, the C-terminus) of a peptide sequence. A secretory signal sequence can be referred to by various names such as, but not limited to, a signal sequence, a targeting signal, a localization signal, a localization sequence, a transit peptide, a leader sequence, a leader peptide, a prepro sequence, a pre sequence, or a secretory signal peptide). A secretory signal sequence can be about 10 to 110 amino acids long (e.g., about 10, 15, 20, 25, 30, 35, 40, 45, 50, 55, 60, 65, 70, 75, 80, 85, 90, 95, 100, 105 or 110 amino acids long). A secretory signal sequence, as a component of a larger polypeptide, can be useful for targeting and can direct the larger polypeptide through a secretory pathway of a cell in which it is synthesized. In some embodiments, the larger polypeptide is cleaved to remove the secretory signal sequence during transit through the secretory pathway. A secretory signal sequence can be endogenous or engineered.

A secretory signal sequence can be synthesized according to the rules established, for example, by von Heinje (Eur. J. Biochem. 133:17-21, 1983; J. Mol. Biol. 184:99-105, 1985; Nuc. Acids. Res. 14:4683-3690, 1986). Examples of secretory signal sequences are shown in Table 2. Any secretory signal sequence known in the art or to those of ordinary skill in the art can be used in the polypeptides and methods described herein.

TABLE 2

| Description | | |
|---|---|---|
| (if applicable) | A secretory signal sequence | SEQ ID NO: |
| TDX1779 and | MDWTWRVFFLLALATGVHS | SEQ ID NO: 24 |
| TDX1780 | | |
| secretory signal | | |
| sequence | | |
| SignalP 3.0 | MDWTWRILFLVAAATGTHA | SEQ ID NO: 25 |
| Erwinia carotovora | MKYLLPTAAAGLLLLAAQPAMA | SEQ ID NO: 26 |
| pectate lyase 2 | | |
| (pelB) leader | | |
| peptide | | |
| Human growth | MATGSRTSLLLAFGLLCLWLQEGSA | SEQ ID NO: 27 |
| hormone signal | | |
| sequence | | |
| otPA pre-pro | MPLLLLLPLLWAGALA | SEQ ID NO: 28 |
| signal sequence | | |
| Human CD33 | MDAMKRGLCCVLLLCGAVFVSLSQEIHAELR | SEQ ID NO: 29 |
| signal sequence | RFRR | |
| PhoA | MKQSTIALALLPLLFTPVKTA | SEQ ID NO: 30 |
| OmpA | MKKTAIAIAVALAGFATVAQA | SEQ ID NO: 31 |
| DsbA | MKKIWLALAGLVLAFSASA | SEQ ID NO: 32 |
| TorT | MRVLLFLLSLFMLPAFS | SEQ ID NO: 33 |
| SufI | MSLSRRQFIQASGIALCAGAVPLKASA | SEQ ID NO: 34 |
| TorA | MNNNDLFQASRRRFLAQLGGLTVAGMLGPS | SEQ ID NO: 35 |
| | LLTPRRATA | |
| N terminal | MAGPATQSPMKLMALQLLLWHSALWTVQEA | SEQ ID NO: 36 |
| secretory signal | | |
| peptide sequence | | |
| human α-1-anti | MMPSSVSWGILLLAGLCCLVPVSLA | SEQ ID NO: 37 |
| trypsin (AAT) | | |
| human Factor IX | MQRVNMIMAESPSLITICLLGYLLSAECTVFLD | SEQ ID NO: 38 |
| (FIX) | HENANKILNRPKR | |
| human Prolactin | MKGSLLLLLVSNLLLCQSVAP | SEQ ID NO: 39 |
| (Prolac) | | |
| human Albumin | MKWVTFISLLFLFSSAYSRGVFRR | SEQ ID NO: 40 |
| (Alb) | | |
| YDR420w | MVSLKIKKLLLVSLLNAIEAYSNDTI | SEQ ID NO: 41 |
| secretory signal | YSTSYNNGIESTPSYSTSAISSTGSSNKENAIT | |
| peptide | SSSETTTMAGDYGESGSTTIMDEQETGTSSQ | |
| | YISVTTTTQ | |
| YBR187w | NGGNMAIKKASLIALLPLFTAAAAAATDAETSN | SEQ ID NO: 42 |
| secretory signal | ESGSSSHLKS | |
| peptide | | |
| YHR139c | MKFTSVLAFFLATLTASATFLYKRQNVTSGGG | SEQ ID NO: 43 |
| secretory signal | TVPIITGGPAVSGSQSNVTTTTLFNSTSTLNIT | |
| peptide | QLYQIATDVNDTLQSESSS | |
| YGR014w | MQFPFACLLSTLVISGSLARASPFDFIFGNGT | SEQ ID NO: 44 |
| secretory signal | QQAQSQSESQGQVSFTNEASQDSSTTSLVT | |
| peptide | AYSQGVHSHQSATIVSATISSLPSTWYDASST | |
| | SQTSVS | |
| YBR078w | MQFKNALTATAILSASALAANSTTSIPSSCSIG | SEQ ID NO: 45 |
| secretory signal | TSATATAQADLDKISQCSTIVGNLTITGDLGSA | |
| peptide | ALASIQEIDGSLTIFNSSSLSSFSADIKKI | |
| YNL300w | MKFSTLSTVAAIAAFASADSTSDGVTYVDVTT | SEQ ID NO: 46 |
| secretory signal | TPQSTTSMVSTVKTTSTPYTTSTIATLSTKSIS | |
| peptide | SQANTTTHEIST | |
| YLR084c | MFVHRLWTLAFPFLVEISKASQLENIKSLLDI | SEQ ID NO: 47 |
| secretory signal | EDNVLPNLNISQNNSNAVQILGGVDALSFYEY | |
| peptide | TGQQNFTKEIGPETSSHGLVYYSNNTYIQLED | |
| | ASDD | |
| YMR008c | MKLQSLLVSAAVLTSLTENVNAMSPNNSYVP | SEQ ID NO: 48 |
| secretory signal | ANVTCDDDINLVREASGLSDNEYEMLKKRDA | |
| peptide | YTKE | |

A polypeptide can comprise about 70%, 75%, 80%, 85%, 90%, 95%, 96%, 97%, 98%, 99% or more sequence identity to one or more polypeptides (e.g., 1, 2, 3, 4, 5, 6, 7, or more) as set forth in SEQ ID NO:1, 2, 3, 4, 5, 6, or 7 (see description of SEQ ID NOs: 5, 6, and 7 below) and can further comprise one or more (e.g., 1, 2, 3, 4, 5, or more) secretory signal sequences, one or more (e.g., 1, 2, 3, 4, 5, or more) epitope tags, or one or more secretory signal sequences and one or more epitope tags. These elements can be present as a fusion protein with one or more linkers between the individual proteins or sequences making up the fusion protein. Alternatively, no linkers can be present between the individual proteins or sequences making up the fusion protein. The elements of the fusion protein (i.e., a polypeptide as set forth in SEQ ID NO:1, 2, 3, 4, 5, 6, or 7, secretory signal sequences, and epitope tags) can occur in any order in the fusion protein.

### VUT Polypeptides

The Rickettsia vitamin uptake transporter VUT family protein is a 200 amino acid, hypothetical protein, predicated to reside on the bacterial cell surface. The proposed function of VUT based on domain homology is consistent with queuosine salvage, a critical requirement for bacterial growth. An example of this protein is GenBank Accession number WP_012150928.1, which is shown in SEQ ID NO:8:

MLFDKIKSKRKGKCMSNINAKFYIPLVSLLGVFIYLLNCFNKISQCSL
VFVFLAITTNIISELYGRKRALIAVALCIIVSFGLLWNFNYYIHGRVI
KGVVFASFVSVLLSTYCSTSIFSQLKPRCSLNTRNFASLIMCAVVDGI
VMSGFFVNVFSTSKVLSIFYKEVLYKCAYSLTVYICIFLVQKVYGNNG
SVRALKNF

In one embodiment, the VUT protein has two regions of interest flanking either end of the protein. PDX21 and PDX39 peptides were derived from the first 38 and last 38 residues, respectively, of the VUT protein. In one embodiment, the PDX21 and PDX39 peptides have point mutations in the cysteine residues. That is, naturally occurring cysteine amino acids are changed to, for example, serine. Furthermore, a naturally occurring methionine can be changed to a cysteine. These changes are underlined below.

(PDX21)
SEQ ID NO: 5
MLFDKIKSKRKGKSMSNINAKFYIPLVSLLGVFIYLLN
(PDX39)
SEQ ID NO: 6
CKEVLYKSAYSLVYISIFLVQKVYGNNGSVRALKNF
(PDX40)
SEQ ID NO: 7
CLFDKIKSKRKGKSMSNINAKFYIPLVSLLGVFIYLLN

PDX40 is PDX21 with the first methionine (M) replaced with cysteine (C) for biochemistry purposes.

These polypeptides are therefore non-naturally occurring and have different properties than naturally occurring polypeptides including, for example, a reduced tendency to form secondary structures and the ability to conjugate to solid phases for testing.

One embodiment provides a purified polypeptide comprising the complete SCA2 protein, the complete VUT protein, PDX6, PDX7, TDX1779, TDX1780, PDX21, PDX39, PDX40 or a fragment thereof. A polypeptide fragment of the complete SCA2 protein, the complete VUT protein, PDX6, PDX7, TDX1779, TDX1780, PDX21, PDX39, PDX40 can be less than about 350, 300, 250, 200, 150, 100, 95, 90, 80, 70, 60, 50, 40, 35, 30, 25, 20, 15, 10 (or any range between about 10 and about 350) total contiguous amino acids. In one embodiment a polypeptide fragment is more than about 10, 15, 20, 25, 30, 35, 40, 50, 60, 70, 80, 90, 95, 100, 150, 200, 250, 300, or 350 total contiguous amino acids of the complete SCA2 protein, the complete VUT protein, PDX6, PDX7, TDX1779, TDX1780, PDX21, PDX39, PDX40. In an embodiment, a polypeptide has less than about 95, 75, 50, or 25 total amino acids and comprises 70%, 80%, 90%, 95% or more sequence identity to a polypeptide as set forth in SEQ ID NO:1, 2, 5, 6, or 7. In an embodiment a polypeptide has less than about 400, 350, 300, 200, or 150 total amino acids and comprises about 70%, 80%, 90%, 95% or more sequence identity to a polypeptide as set forth in SEQ ID NO: 3 or 4.

For example, SEQ ID NO:1 is 48 amino acids in length. In an embodiment, the polypeptide can comprise additional amino acids to extend the polypeptide to a length of, for example 95 amino acids. The extra or additional amino acids can be, for example, labels, tags, additional R. rickettsii amino acids, amino acids unrelated to R. rickettsii, amino acids that can be used for purification, amino acids that can be used to increase solubility of the polypeptide, amino acids to improve other characteristics of the polypeptide, or other amino acids. In an embodiment, the additional amino acids are not R. rickettsii amino acids. In this example, the 95 amino acid long polypeptide has about 70%, 80%, 90%, 95% or more sequence identity to a polypeptide as set forth in SEQ ID NO:1 over the 48 consecutive amino acids of SEQ ID NO: 1, while the remaining 47 amino acids of the 95 amino acid polypeptide can have, e.g., no sequence identity to SEQ ID NO:1.

In another non-limiting example, a polypeptide having less than 75 total amino acids and comprising 90% or more sequence identity to a polypeptide as set forth in SEQ ID NO:2 can be a polypeptide having 70 amino acids. The 70 amino acid long polypeptide can have 90% or more sequence identity to the polypeptide as set forth in SEQ ID NO:2 over the 38 consecutive amino acids of SEQ ID NO:2, while the remaining 32 amino acids of the 70 amino acid polypeptide can have, e.g., no sequence identity to SEQ ID NO:2.

In another non-limiting example, a polypeptide having less than 350 total amino acids and comprising 90% or more sequence identity to a polypeptide as set forth in SEQ ID NO:3 can be 300 amino acids in length. The 300 amino acid long polypeptide can have 90% or more sequence identity to the polypeptide as set forth in SEQ ID NO:3 over the 172 consecutive amino acids of SEQ ID NO:3, while the remaining 128 amino acids of the 300 amino acid polypeptide can have, e.g., no sequence identity to SEQ ID NO:3.

The fact that a polypeptide (e.g., SEQ ID NOs: 1-7) is smaller than a full length a R. rickettsii polypeptide can be important because smaller polypeptides can have greater specificity and/or sensitivity than full length polypeptides in detection or diagnostic assays. Additionally, these smaller polypeptides can be less expensive to manufacture and can be obtained at greater purity than full length polypeptides.

In one embodiment, a polypeptide or fragment thereof is non-naturally occurring. That is, a polypeptide or fragment comprises 1, 2, 3, 4, 5, 10, 20, 30, 40, 50, 75 or more non-naturally occurring amino acids. In an embodiment, the non-naturally occurring amino acids can provide a beneficial property such as increased solubility of the polypeptide or increased sensitivity or increased specificity of the polypeptide in assays.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NOs: 1-7) aligned for comparison purposes is at least 50, 60, 70, or 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence (e.g., SEQ ID NOs: 1-7).

Polypeptides that are sufficiently similar to polypeptides described herein (e.g., SCA2 or VUT polypeptides) can be used herein. Polypeptides that are about 90, 91, 92, 93, 94 95, 96, 97, 98, 99, 99.5% or more identical to polypeptides described herein can also be used herein.

A polypeptide variant differs by about, for example, 1, 2, 3, 4, 5, 6, 7, 8, 9, 10, 20, 30, 40, 50, 60, 70, 80, 90, or more amino acid residues (e.g., amino acid additions, substitutions, or deletions) from a peptide shown SEQ ID NOs: 1-7 or a fragment thereof. Where this comparison requires alignment, the sequences are aligned for maximum homology. The site of variation can occur anywhere in the polypeptide. In one embodiment, a variant has about 70%, 75%, 80%, 85%, 90%, 95%, 96%, 97%, 98%, 99% sequence identity to the original polypeptide.

Variant polypeptides can generally be identified by modifying one of the polypeptide sequences described herein and evaluating the properties of the modified polypeptide to determine if it is a biological equivalent. A variant is a biological equivalent if it reacts substantially the same as a polypeptide described herein in an assay such as an immunohistochemical assay, an enzyme-linked immunosorbent assay (ELISA), a turbidimetric immunoassay, a particle-enhanced turbidimetric immunoassay, a particle-enhanced turbidimetric immunoassay, a radioimmunoassay (RIA), immunoenzyme assay, a western blot assay, or other suitable assay. In other words, a variant is a biological equivalent if it has 90-110% of the activity of the original polypeptide. In one embodiment, the assay is a competition assay wherein the biologically equivalent polypeptide is capable of reducing binding of the polypeptide described herein to a corresponding reactive antigen or antibody by about 80%, 95%, 99%, or 100%. An antibody that specifically binds a corresponding polypeptide also specifically binds the variant polypeptide.

Variant polypeptides can have one or more conservative amino acid variations or other minor modifications and retain biological activity, i.e., are biologically functional equivalents to PDX6, PDX7, TDX1779, TDX1780, PDX21, PDX39, PDX40 or a fragment thereof. Variant polypeptides can have labels, tags, additional R. rickettsii amino acids, amino acids unrelated to R. rickettsii, amino acids that can be used for purification, amino acids that can be used to increase solubility of the polypeptide, amino acids to improve other characteristics of the polypeptide, or other amino acids. In an embodiment, the additional amino acids are not R. rickettsii amino acids.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art. A variant polypeptide can also be chemically synthesized.

Variant polypeptides can have conservative amino acid substitutions at one or more predicted nonessential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. In one embodiment a polypeptide has about 1, 2, 3, 4, 5, 10, 20 or fewer conservative amino acid substitutions.

A polypeptide can be a fusion protein, which can contain other amino acid sequences, such as amino acid linkers, amino acid spacers, signal sequences, TMR stop transfer sequences, transmembrane domains, as well as ligands useful in protein purification, such as glutathione-S-transferase, histidine tag (e.g., about 6, 7, 8, 9, 10, or more His residues), and staphylococcal protein A, or combinations thereof. In an embodiment, a polypeptide comprises one or more epitope tags, such as FLAG (for example, DYKDDDDK; SEQ ID NO:16), HA (YPYDVPDYAC; SEQ ID NO:9), myc (EQKLISEEDLC; SEQ ID NO:10), V5 (GKPIPNPLLGLDST; SEQ ID NO:11), E-tag (GAPVPYPDPLEPR; SEQ ID NO:12), VSV-g (YTDIEMNRLGK; SEQ ID NO:13), 6xHis (HHHHHHH; SEQ ID NO:14), and HSV (QPELAPEDPEDC; SEQ ID NO:15).

An antibody, such as a monoclonal antibody, can specifically bind to an epitope tag and be used to purify a polypeptide comprising the epitope tag.

A fusion protein can comprise two or more different amino acid sequences operably linked to each other. A fusion protein construct can be synthesized chemically using organic compound synthesis techniques by joining individual polypeptide fragments together in fixed sequence. A fusion protein can also be chemically synthesized. A fusion protein construct can also be expressed by a genetically modified host cell (such as E. coli) cultured in vitro, which carries an introduced expression vector bearing specified recombinant DNA sequences encoding the amino acids residues in proper sequence. The heterologous polypeptide can be fused, for example, to the N-terminus or C-terminus of a polypeptide. More than one polypeptide can be present in a fusion protein. Fragments of polypeptides can be present in a fusion protein. A fusion protein can comprise, e.g., one, two, three, four, five, six, seven or more of PDX6, PDX7, TDX1779, TDX1780, PDX21, PDX39, PDX40, fragments thereof, or combinations thereof. Polypeptides can be in a multimeric form. In other words, a polypeptide can comprise two or more copies (e.g., two, three, four, five, six, seven or more) of PDX6, PDX7, TDX1779, TDX1780, PDX21, PDX39, PDX40, fragments thereof, or a combination thereof. A polypeptide can include, e.g., a fusion protein of two, three, four, five, six, seven or more polypeptides having about 70%, 75%, 80%, 85%, 90%, 95%, 96%, 97%, 98%, 99% or more sequence identity to SEQ ID NO:1, 2, 3, 4, 5, 6, or 7; or a fusion protein of at least two polypeptides having about 70%, 75%, 80%, 85%, 90%, 95%, 96%, 97%, 98%, 99% sequence identity to SEQ ID NO: 1, 2, 3, 4, 5, 6, or 7. A polypeptide can be a fusion protein that can include one or more linkers between the individual proteins making up the fusion protein (i.e., SEQ ID NO:1, 2, 3, 4, 5, 6, or 7). Alternatively, no linkers can be present between the individual proteins making up the fusion protein. A fusion polypeptide can contain other amino acid sequences, such as amino acid linkers, amino acid spacers, signal sequences, TMR stop transfer sequences, transmembrane domains, as well as ligands useful in protein purification, such as glutathione-S-transferase, histidine tag, epitope tags, and staphylococcal protein A, or combinations thereof.

A still further component of a fusion protein can be a secretory (signal) sequence. These sequences can allow for secretion of the fusion protein from the host cell during expression. The secretory (signal) sequence can be that of the heterologous protein being produced, if it has such a sequence, or can be derived from another secreted protein (e.g., t-PA), or synthesized de novo. The polynucleotide sequence encoding the secretory (signal) sequence can be operably linked to fusion protein DNA sequence, i.e., the two sequences are joined in the correct reading frame and positioned to direct the newly synthesized polypeptide into the secretory pathway of the host cell. Polynucleotide sequences encoding secretory (signal) sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

In an embodiment a polypeptide as described herein is present in an immunocomplex with one or more antibodies or specific binding fragments thereof that specifically bind to a polypeptide as set forth in SEQ ID NO:1, 2, 3, 4, 5, 6, or 7. The one or more antibodies or specific binding fragments thereof can be anti-R. rickettsii antibodies or specific binding fragments thereof.

Polypeptides can be lyophilized, desiccated, or dried, for example freeze-dried. A lyophilized polypeptide can be obtained by subjecting a preparation of the polypeptides to low temperatures to remove water from the sample. A desiccated polypeptide composition can be obtained by drying out a preparation of the polypeptides by removal of water. A dried polypeptide preparation can refer to a polypeptide preparation that has been air dried (e.g., lyophilized).

### Labels

One or more polypeptides described herein (including detector polypeptides) can be conjugated to one or more labels or tags. A label or tag can be coupled directly or indirectly to the desired component of the assay (e.g., an antibody, polypeptide, or support). For example, polypeptides can be conjugated to a fluorophore, an enzyme, a chemiluminescent moiety, a radioactive moiety, an organic dye, a small molecule, a polypeptide or functional fragment thereof. Examples of fluorophores include fluorescent dyes like phycoerytherin (PE), fluorescein isothiocyanate (FITC), tetramethylrhodamine (TRITC), BODIPY, Texas red, rhodamine and AlexaFluor® dyes. Fluorescent dyes can also comprise fluorescence resonance energy transfer (FRET)-dyes or time-resolved (TR)-FRET dyes. Fluorophore labels can comprise fluorescent proteins such as green fluorescent protein (GFP), enhanced green fluorescent protein, and cyan fluorescent protein (CFP). Examples of enzyme labels include hydrolases, such as phosphatases including alkaline phosphatase, esterases and glycosidases, or oxidoreductases, peroxidases such as horseradish peroxidase, and others commonly used in enzyme-linked immunosorbent assays, ELISAs. When a substrate such as 3,3′5,5′-Tetramethylbenzidine (TMB), 3,3′-Diaminobenzidene (DAB), or 2,2′-azino-bis(3-ethylbenzothiazoline-6-sulphonic acid) (ABTS) are applied to HRP, a colored (chromogenic) or light (chemiluminescent) signal is produced. Radioactive moiety labels can be, for example, ³H, ¹²⁵I, ³⁵S, ¹⁴C, or ³²P. Small molecule labels include, for example, resins such as agarose beads and magnetic beads such as fluorescently labeled magnetic beads or barcoded magnetic beads (e.g., barcoded magnetic beads available from Applied BioCode of Santa Fe Springs, CA), Dynabeads™, colored glass or plastic (e.g., polystyrene, polypropylene, latex, etc.) beads, and nanoparticles such as colloidal gold. Polypeptide or functional fragment labels include, for example, avidin, streptavidin or NeutrAvidin, which have an affinity for biotin, hemagglutinin (HA), glutathione-S-transferase (GST), or c-myc.

Detection of labels or tags can be done using many different methods. For example, a radioactive label can be detected using a scintillation counter, photographic film as in autoradiography, or storage phosphor imaging. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Simple colorimetric labels can be detected by observing the color associated with the label. When pairs of fluorophores are used in an assay, they may have distinct emission patterns (wavelengths) so that they can be easily distinguished.

While components described herein (e.g., polypeptides, antibodies and specific binding fragments thereof) can comprise a label, a label is not necessary for detection of polypeptide/antibody complexes since many options exist for label-free detection including, for example surface plasmon resonance, bio-layer interferometry, and grating-coupled interferometry detection assays.

### Supports

In an embodiment, a component of an assay (e.g., a polypeptide or antibody or specific binding fragment thereof) can be immobilized to a support. A support is any material that is appropriate for or that can be modified to be appropriate for attachment of one or more polypeptides, antibodies, or specific binding fragments as described herein. Examples of supports include glass and modified or functionalized glass, plastics (including acrylics, polystyrene, methylstyrene, polyurethanes, Teflon®, etc.), paramagnetic materials, thoria sol, carbon graphite, titanium oxide, latex or cross-linked dextrans such as Sepharose, cellulose polysaccharides, nylon or nitrocellulose, ceramics, resins, silica or silica-based materials including silicon and modified silicon, carbon metals, inorganic glasses, optical fiber bundles, and a variety of other polymers. In an embodiment a support can be located in a microtiter well plate (e.g., a 96-well, 384-well or 1536-well plate). In an embodiment, a support can be located within a flow cell or flow cell apparatus (e.g., a flow cell on a protein chip). A support can be a solid support.

In an embodiment, a support can be a bead such as a magnetic barcoded bead, microsphere, particle, membrane, chip, slide, well, or test tube. Beads include microspheres or particles, which can be small, discrete, non-planar particles in the micrometer or nanometer dimensions. A bead can be spherical or irregular. A bead can be porous. In an embodiment, a support can comprise a patterned surface suitable for immobilization of polypeptides in an ordered pattern (e.g., a protein chip).

In an embodiment one or more polypeptides as described herein can be immobilized to a support via a linker molecule. One or more polypeptides can be conjugated to a support using any suitable methodology. In an embodiment one or more polypeptides are conjugated to a support using a conjugation reagent, including covalent and non-covalent conjugation reagents. Covalent conjugation reagents can include any chemical or biological reagent that can be used to covalently immobilize a polypeptide on a surface. Covalent conjugation reagents include, for example, a carboxyl-to-amine reactive group such as carbodiimides such as EDC or DCC, an amine reactive group such as N-hydroxysuccinimide (NHS) ester or imidoesters, a sulfhydryl-reactive crosslinker such as maleimides, haloacetyls, or pyridyl disulfides, carbonyl-reactive crosslinker groups such as, hydrazides or alkoxyamines, a photoreactive crosslinker such as aryl azides or dizirines, or a chemoselective ligation group such as a Staudinger reaction pair. Non-covalent immobilization reagents can include any chemical or biological reagent that can be used to immobilize a polypeptide non-covalently on a surface, such as affinity tags such as biotin or capture reagents such as streptavidin or anti-tag antibodies, such as anti-His6 or anti-Myc antibodies.

### Methods of Detection and Diagnosis

In an embodiment an assay method can comprise contacting a test sample with one or more polypeptides under conditions suitable for formation of complexes between polypeptides and antibodies or specific binding fragments thereof. The one or more polypeptides can comprise, for example, about 70%, 75%, 80%, 85%, 90%, 95%, 96%, 97%, 98%, 99% sequence identity to SEQ ID NO:1, 2, 3, 4, 5, 6, or 7, fragments, or variants of these polypeptides as described herein. Complexes of polypeptides and antibodies or specific binding fragments thereof can be detected. In an embodiment, complexes of polypeptides and antibodies or specific binding fragments can also comprise one or more labels or tags. If complexes are detected, then the sample contains anti-Rickettsia rickettsii antibodies.

In an embodiment, a method comprises diagnosing a disease caused by Rickettsia rickettsii in a subject. A test sample can be contacted with one or more polypeptides comprising about 70%, 75%, 80%, 85%, 90%, 95%, 96%, 97%, 98%, 99% sequence identity to SEQ ID NO:1, 2, 3, 4, 5, 6, or 7, fragments, or variants as described herein. Complexes of anti-Rickettsia rickettsii antibodies or specific binding fragments thereof and the one or more polypeptides are then detected. The subject can have been infected with Rickettsia for less than about 14, 13, 12, 11, 10, 9, 8, 7, 6, 5, 4, 3, or 2 days. The amount of anti-Rickettsia rickettsii antibodies or specific binding fragments thereof in the sample can be determined. The amount of the complexes in the sample can be compared to a control sample or control standard, wherein elevated levels of the complexes as compared to the control sample or control standard is an indication of a disease caused by R. rickettsii. Where the complexes are detected a treatment can be administered to the subject. A subject can be human or a non-human mammal, such as a dog, horse, cow, or cat. A test sample can be, for example, blood, plasma, serum, lymph fluid, or any other body fluid sample.

In an embodiment specific binding between one or more polypeptides described herein and one or more anti-Rickettsia rickettsii antibodies or specific binding fragments thereof are detected using a secondary antibody or a specific binding fragment thereof. In an example, a secondary antibody can be used to detect the specific binding. For example, a secondary antibody or specific binding fragment thereof is capable of binding the anti-Rickettsia rickettsii antibodies or specific binding fragments thereof. In an embodiment a secondary antibody is an anti-species antibody such as a rabbit anti-dog or rabbit anti-human antibody. In an embodiment, the secondary antibody is covalently or non-covalently bound to a label or tag that can be used for detection of polypeptide/anti-Rickettsia rickettsii antibody/secondary antibody complex. In one example, a secondary antibody can be conjugated to biotin or other label or tag. A streptavidin conjugate (in the case of a biotin label), such as streptavidin fluorochrome conjugate, can be used to detect the polypeptide/anti-Rickettsia rickettsii antibody/secondary antibody complex.

In an embodiment specific binding between one or more polypeptides described herein and one or more anti-Rickettsia rickettsii antibodies or specific binding fragments thereof are detected using one or more detector polypeptides that specifically bind anti-Rickettsia rickettsii antibodies or specific binding fragments thereof. In an example, the one or more detector polypeptides comprise about 70%, 75%, 80%, 85%, 90%, 95%, 96%, 97%, 98%, 99% sequence identity to a polypeptide as set forth in SEQ ID NO:1, 2, 3, 4, 5, 6, or 7, fragments thereof, or variants as described herein. In an embodiment, the one or more detector polypeptides are covalently or non-covalently bound to a label or tag that can be used for detection of polypeptide/anti-Rickettsia rickettsii antibody/detector polypeptide complex. In one example, a detector polypeptide can be conjugated to a label or tag such as biotin. A streptavidin conjugate (in the case of a biotin label), such as streptavidin fluorochrome conjugate can be used to detect the polypeptide/anti-Rickettsia rickettsii antibody/detector polypeptide complex.

The detection of specific binding between one or more polypeptides described herein and one or more antibodies or specific binding fragments thereof can be completed using any suitable method, for example, an immunoassay such as a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, or a western blot assay. Other assay methods that can be used to detect antibodies and/or antibody/polypeptide complexes include, for example surface plasmon resonance (SPR), isothermal titration calorimetry (ITC), microscale thermophoresis (MST), biolayer interferometry, or grating-coupled interferometry.

Antibodies described herein can be a polyclonal antibody, a monoclonal antibody, a single chain antibody (scFv), or a specific binding fragment of an antibody. Antibody specific binding fragments are one or more portions of an intact antibody that comprises the antigen binding site or variable region of an intact antibody, where the portion is free of the constant heavy chain domains of the Fc region of the antibody. Examples of antibody fragments that bind to antigens include Fab, Fab′, Fab′-SH, F(ab′) 2 and Fv fragments. An antibody described herein can be any class of antibody that includes, for example, IgG, IgM, IgA, IgD and IgE.

“Specifically binds” or “specific for” means that a first antigen, e.g., a polypeptide as shown in SEQ ID NOs: 1-7, fragments thereof, or variants as described herein, recognizes and binds to an anti-R. rickettsia antibody or specific binding fragment thereof with greater affinity than other non-specific molecules. A non-specific molecule is an antigen that shares no common epitope with the first antigen. In an embodiment, a non-specific molecule is not a R. rickettsia polypeptide and is not related to R. rickettsia. In an embodiment, a non-specific molecule is not derived from a Rickettsia organism. For example, an antibody raised against a first antigen (e.g., R. rickettsia) to which it binds more efficiently than to a non-specific antigen can be described as specifically binding to the first antigen. A polypeptide specifically binds to an anti-R. rickettsia antibody or specific binding fragment when it binds with a binding affinity (K_{D}) of 10⁻⁶ M or less. In an embodiment, a polypeptide specifically binds to an anti-R. rickettsia antibody or specific binding fragment when it binds with an affinity (K_{D}) of 2×10⁻⁶ M or less. In an embodiment, a polypeptide specifically binds to an anti-R. rickettsia antibody or specific binding fragment when it binds with an affinity (K_{D}) of at least 10⁻⁶ M, 10⁻⁷ M, 10⁻⁸ M, 10⁻⁹ M or less. In certain embodiments, the affinity is measured by surface plasmon resonance or KinExA assay. In an embodiment, the methods specifically detect Rickettsia rickettsii and do not detect other Rickettsia species such as R. conorii, R. africae, R. japonica, R. phillipii, R. parkeri, R. massiliae, R. felis, R. typhi, R. prowazekii, R. bellii, or combinations thereof.

### Kits

A kit can comprise one or more of the polypeptides described herein. A kit can also include a tag, label, or conjugate for detection. One or more polypeptides can be covalently or non-covalently immobilized on a support.

For example, a label of the kit can include a fluorophore, an enzyme, a chemiluminescent moiety, a radioactive moiety, an organic dye, a small molecule, a polypeptide or functional fragment thereof. In some embodiments, a label of the kit includes phycoerytherin. In some embodiments, a label of the kit includes fluorescein isothiocyanate. In some embodiments, a label is conjugated to a secondary antibody or detector polypeptide.

A kit can include a positive control. In some embodiments, a positive control can be a sample containing a detectable amount of anti-R. rickettsia antibodies. In an embodiment, a positive control can be obtained from a diseased subject who has anti-R. rickettsia antibodies. In an embodiment, a positive control can comprise anti-R. rickettsia antibodies synthesized in vitro or otherwise obtained. In an embodiment, a kit can include a negative control. A negative control can be a sample containing no detectable amount of anti-R. rickettsia antibodies. In some embodiments, a negative control can be obtained from a healthy control individual or can be synthesized in vitro. For example, a negative control can include water or buffer.

In some embodiments a kit can include a standard curve for determining an amount of anti-R. rickettsia antibodies in a sample. Anti-Rickettsia rickettsia antibodies can be used to create a standard curve for an assay. A standard curve can be used to determine the concentration of anti-Rickettsia rickettsia antibodies in a sample. A standard curve is obtained by relating a measured quantity to the concentration of the anti-Rickettsia rickettsia antibodies in “known” samples, i.e., standards of known concentration. These standards provide a reference to determine unknown concentrations of anti-R. rickettsia antibodies in a sample. The amounts of standards can span the whole range of concentrations expected to be found in the “unknown” or “test” sample concentration.

A kit can further include one or more assay reagents that facilitate binding of the one or more polypeptides to anti-Rickettsia rickettsii antibodies or specific binding fragments thereof. The assays reagents are substances, mixtures, materials, or components that are useful to carry out an intended purpose of the kit. Reagents can include, for example, a conjugation reagent, a buffer, standard, positive control, label, a sample collection device, instructions and the like.

A kit can include one or more buffers, such as a wash buffer. A wash buffer can include, for example, tris(hydroxymethyl) aminomethane (Tris)-based buffers like Tris-buffered saline (TBS) or phosphate buffers like phosphate-buffered saline (PBS). Wash buffers can be composed of, for example, detergents, such as ionic or non-ionic detergents. In some embodiments, a wash buffer can be a PBS buffer at about pH 7.0, 7.2, 7.4, 7.6, 7.8 including Tween™20 (polysorbate) at about 0.001, 0.01, 0.02, 0.03, 0.04, 0.05, 0.06, 0.07, 0.08% or more.

A kit can include a dilution buffer. Dilution buffers include, for example, a carrier protein such as bovine serum albumin (BSA) and a detergent such as Tween®20 (polysorbate).

A kit can include a detection or assay buffer. A detection or assay buffer can be, for example, a colorimetric detection or assay buffer, a fluorescent detection or assay buffer, or a chemiluminescent detection or assay buffer. Colorimetric detection or assay buffers include, for example, PNPP (p-nitrophenyl phosphate), ABTS (2,2′-azino-bis(3-ethylbenzothiazoline-6-sulphonic acid)) or OPD (o-phenylenediamine). Fluorescent detection or assay buffers include QuantaBlu® or QuantaRed® (Thermo Scientific, Waltham, MA). Chemiluminescent detection or assay buffers can include luminol or luciferin. Detection or assay buffers can also include a trigger such as H₂O₂ and a tracer such as isoluminol-conjugate.

A kit can include a stop solution. Stop solutions of can terminate or delay the further development of the detection reagent and corresponding assay signals. Stop solutions can include, e.g., low-pH buffers (e.g., glycine-buffer, pH 2.0), chaotrophic agents (e.g., guanidinium chloride, sodium-dodecylsulfate (SDS)), reducing agents (e.g., dithiothreitol, β-mercaptoethanol), or the like.

A kit provided can include a device for collecting a biological sample, such as collection tubes, columns, swabs, syringes, and needles. A kit can include instructions for using the components of the kit. The instructions can provide details regarding protocols and analytical techniques.

Components of a kit can be in any physical state. For example, one or more of the components can be lyophilized, in aqueous solution, or frozen.

A kit of can be designed for specific assay technologies. For example, a kit can be an immunoassay kit, a competitive immunoassay kit, a sandwich immunoassay kit, an enzyme-linked immunosorbent assay (ELISA) kit, an immunohistochemical assay kit, a turbidimetric immunoassay kit, a particle-enhanced turbidimetric immunoassay kit, a radioimmunoassay (RIA) kit, a fluorescent immunosorbent assay (FIA) kit, a multiplex immunoassay kit, a protein/peptide array immunoassay kit, a solid phase radioimmunoassay (SPRIA) kit, an indirect immunofluorescence assay (IIF) kit, a chemiluminescent immunoassay (CIA) kit, a particle based multianalyte test (PMAT) kit, a dot blot assay kit, or a western blot assay kit, a surface plasmon resonance (SPR) kit, an isothermal titration calorimetry (ITC) kit, a microscale thermophoresis (MST) kit, a biolayer interferometry kit, or grating-coupled interferometry kit. In an embodiment, an ELISA kit can include, for example, a wash buffer, sample diluent, a secondary antibody, a secondary antibody-enzyme conjugate, a detector polypeptide, a labeled detector polypeptide, a detection reagent, and a stop solution. In an embodiment a dot blot kits can include, for example, a wash buffer, sample diluent, a secondary antibody-enzyme conjugate, a detection reagent, and a stop solution. In some embodiments, a chemiluminescent immunoassay kit can include a wash buffer, a sample diluent, a tracer (e.g., isoluminol-conjugate) and a trigger (e.g., H₂O₂). In an embodiment, a multiplex kit can include, for example, a wash buffer, a sample diluent, and a secondary antibody-enzyme conjugate.

### Methods of Treatment

Once detected or diagnosed, RMSF can be treated with, for example, antibiotic drugs, protein or peptide drugs, nucleic acid-based drugs, anti-inflammatory drugs, other drugs, immunomodulatory treatments, alternative therapies, or combinations thereof.

For example, RMSF can be treated with a number of antibiotics, for example, doxycycline (Monodox, Vibramycin), tetracycline (oxytetracycline), azithromycin, chloramphenicol (Chloromycetin), trovafloxacin (Trovan), minocycline derivatives (see e.g., US20160030452), and beta-lactamase inhibitors (see e.g., US20130023512). Doxycycline can be administered to adults at about 50, 100, 150, 200 mg every 6 hours, every 12 hours, once a day, or once every other day for 2, 4, 6, 8, 10, 12, 14 days or more. Children under 45 kg can be administered about 1.0, 2.0, 2.2, 3.0, 4.0 mg/kg body weight or more every 6 hours, every 12 hours, once a day, or once every other day for 2, 4, 6, 8, 10, 12, 14 days or more.

Protein or peptide drugs suitable for RMSF treatment include, for example, isolated chimeric outer surface protein A (OspA) (see e.g., US20180296656, US20160333056), μ-AApeptides (see e.g., US20150274782), monoclonal antibodies specific for flagellin (see e.g., US20100239583), peptide compounds for treating non-inflammatory musculoskeletal pain or osteoarthritic pain (see e.g., US20080280835), and heterologous recombinant Sca5/OmpB protein-based vaccines.

RMSF can be treated with nucleic acid based drugs, derivatives thereof (see e.g., US2012002104, US20090324584), and lipid-modified nucleic acids (see e.g., US20070280929).

RMSF can be treated with anti-inflammatory drugs, for example, cellulose sulfate polymers or chitosan (negatively charged) polymers (see e.g., US20090298792), platelet activating factor (PAF) inhibitors and antioxidants that interferes with the arachidonic acid cascade (see e.g., US20040022879), and prednisolone or other steroids at anti-inflammatory or immunosuppressive dosages.

RMSF can be treated with, for example, immunomodulatory compounds (see e.g., US20110184025), such as enantiomerically pure 4-(amino)-2-(2,6-dioxo (3-piperidyl))-isoindoline-1,3-dione or 3-(4-amino-1-oxo-1,3-dihydro-isoindol-2-yl)-piperidine-2,6-dione) administered alone or with an antibiotic, functional immunostimulatory protein CD40L, constitutively active TLR4 (caTLR4), functional immunostimulatory protein CD70 (see e.g., US20170000881), and/or intravenous immunoglobulin therapy.

RMSF can also be treated with a number of additional drugs that encompass pharmaceutical and/or dietary supplements of copper (I) complex with glycine, pyruvate, and/or succinic acid (see e.g., US20180071336), isothiocyanate functional surfactants (see e.g., US 2018/0311202), isooxazoline compositions used as antiparasitics such as 4-(isoxazolinyl)-benzamides (e.g., substituted 4-(5-(halomethyl)-5-phenyl-isoxazolin-3-yl)-benzamides) and 4-(isoxazolinyl)-benzothioamides (e.g., substituted 4-(5-(halomethyl)-5-phenyl-isoxazolin-3-yl)-benzothioamides) (see e.g., US20180155301), pyrazolo[1,5-a][1,3,5]triazine derivatives and pharmaceutically acceptable salts thereof (see e.g., US20150111873), topically administering an electrolytic acid water comprising free chlorine (see e.g., US20130259955), proteasome inhibitors (see e.g., US20110172285), polymorphs of the hydrochloride salt(S)-3-aminomethyl-7-(3-hydroxy-propoxy)-3H-benzo[c][1,2]oxaborol-1-ol (see e.g., US20110152217), pyridinylamines (see e.g., US20090196912), heterobicyclic compounds (such as 4,5,6,7-tetrahydrobenzo[b]thiophene-3-carboxylic acid amides, 4,7-dihydro-5H-thieno[2,3-c]thiopyran 3-carboxylic acid amides, 4,7-dihydro-5H-thieno[2,3-c]pyran-3-carboxylic acid amides, or benzo[b]thiophene-3-carboxylic acid amides) and pharmaceutically acceptable salts (see e.g., US20070275962), pyrazine derivatives (see e.g., US20080194574), aromatic amidine inhibitors of trypsin-like proteases (such as bis(5-amidino-2-benzimidazolyl)-methane, 1,2-bis(5-amidino2-benzimidazolyl) ethane, 1,5-bis(5-amidino-2-benzimidazolyl) pentane, and 5-amidinoindole), and/or ATP synthase inhibitor for treating diseases and conditions associated with mitochondrial function (see e.g., US20090275099).

RMSF can also be treated with antimicrobial compounds as described in US20140256826 or compositions of high penetration compositions (HPC) or high penetration prodrugs (HPP) of antimicrobials and antimicrobial-related compounds (see e.g., US20130018029).

RMSF can also be treated with, for example, sonic lysates of recombinant E. coli, anti-TNF-alpha inhibitor, and/or nuclear factor kappa B.

Alternative therapies for treatment of RMSF include increased fluid intake, decreased glucose and sugar intake, decreased high-fat foods that slow digestion, small meals, mild or bland food diet, probiotic products such a kombucha or miso soup, magnesium, potassium, calcium, chlorella, spirulina, essential oil mixtures (peppermint oil, cypress oil, and coconut oil, for example), ginger, chamomile tea, vitamin B6, lemon, a phenolic-rich maple syrup extract administered with at least one antibiotic (see e.g., US20160339071), a cool compress to the forehead or back of neck, acupuncture, and/or sitting upright after eating.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1: SCA2 Polypeptides

An autochaperone domain (AC) of the extracellular protein SCA2, was discovered that comprises a stretch of amino acids that is only present in R. rickettsii strains. The entire unique region (boxed in FIG. 3) was named PDX6. A slightly truncated boxed region (FIG. 3) was named PDX7.

Two recombinant fusion proteins were made by fusion of 3 PDX6 repeats (“TDX1779) or 5 PDX6 repeats (“TDX1780”). See FIG. 3. In this example TDX1779 and TDX1780 additionally comprise an epitope tag at the carboxy termini of the polypeptides and a secretory (signal) sequence at the n termini of the polypeptides. PDX7, TDX1779 and TDX1780 were coated to barcoded magnetic beads (BMBs). PDX7 was linked onto an amino activated bead with an SM (PEG) 12 linker (ThermoFisher). TDX1779 and TDX1780 were linked onto carboxy activated beads with an EDAC linker. The PDX, TDX1779, and TDX1780 linked beads were tested for reactivity on both indirect and direct formats.

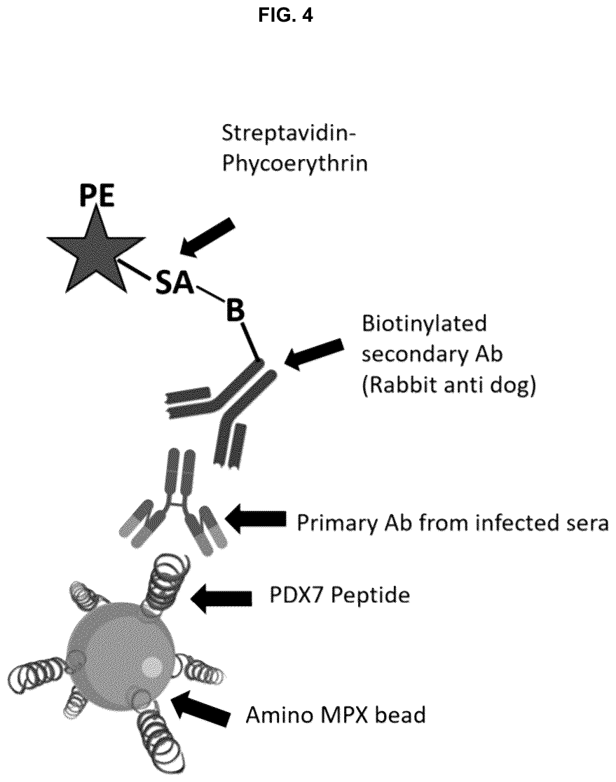

PDX7, TDX1779 and TDX1780 were tested for reactivity in multiplex using an indirect (anti-species antibody) format. A diagram of the indirect methodology is shown in FIG. 4. Briefly, a primary antibody that specifically binds one or more epitopes present in PDX7, TDX1779, or TDX1780 (from, for example, an infected dog) can specifically bind to a PDX7, TDX1779 and TDX1780 peptide coated bead. A labeled (e.g., biotinylated) secondary antibody (e.g., an anti-species antibody, in this case a rabbit anti-dog antibody) specifically binds to the primary antibody. Streptavidin-phycoerythrin can be used to detect the bead-peptide-primary antibody-labeled secondary antibody complex.

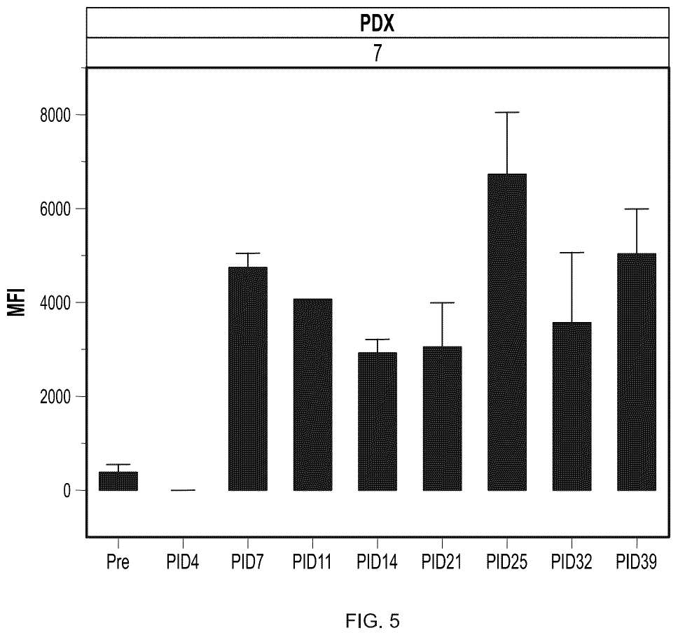

PDX7 was conjugated to amino activated beads and incubated with pooled serum from R. rickettsia experimentally infected canines. Detection of the antibody binding was reported using biotinylated rabbit anti-canine IgG. The results are shown in FIG. 5 (PID means post-infection day). PDX7 was able to detect infection by the seventh day post infection.

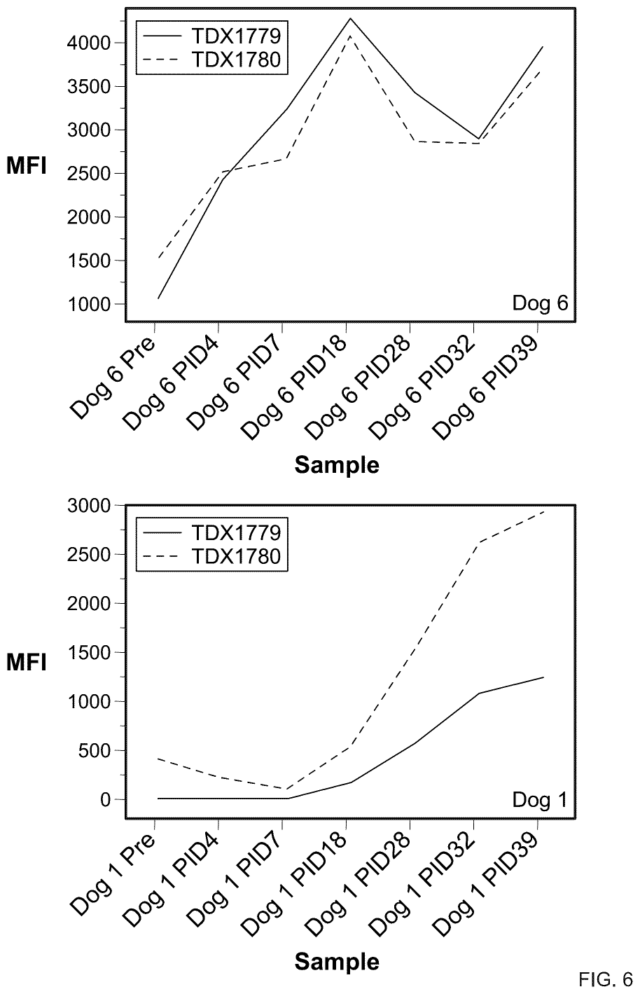

TDX1779 and TDX1780 were coupled to carboxy beads and incubated against serum from R. rickettsii experimentally infected canines. Detection of the antibody binding was reported using biotinylated rabbit anti canine IgG. FIG. 6 shows TDX1779 and 1780 reactivity to experimental sera in the indirect anti-species format. TDX1779 and TDX1780 were able to detect antibodies in about 7 days after infection to varying extents depending on the dog tested.

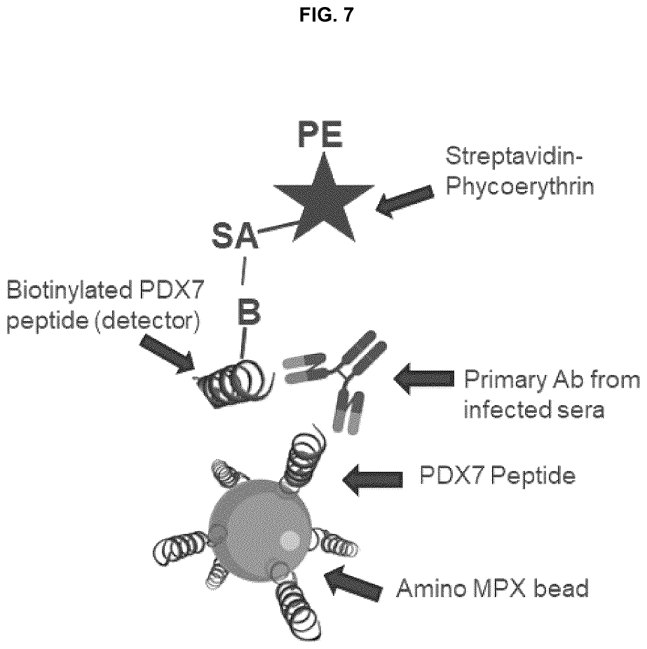

PDX7, TDX1779 and TDX1780 were also tested for reactivity in a direct (e.g., biotinylated analyte) format. A diagram of the direct methodology is shown in FIG. 7. Briefly, a primary antibody that specifically binds one or more epitopes present in PDX7, TDX1779, or TDX1780 (from, for example, an infected dog) can specifically bind to a PDX7, TDX1779 and TDX1780 peptide coated bead. A detector polypeptide (e.g., a PDX7, TDX1779 and TDX1780 polypeptide) can also specifically bind to the primary antibody. The detector polypeptide can be labeled with, for example, biotin. Streptavidin-phycoerythrin can be used to detect the bead-peptide-primary antibody-detector complex.

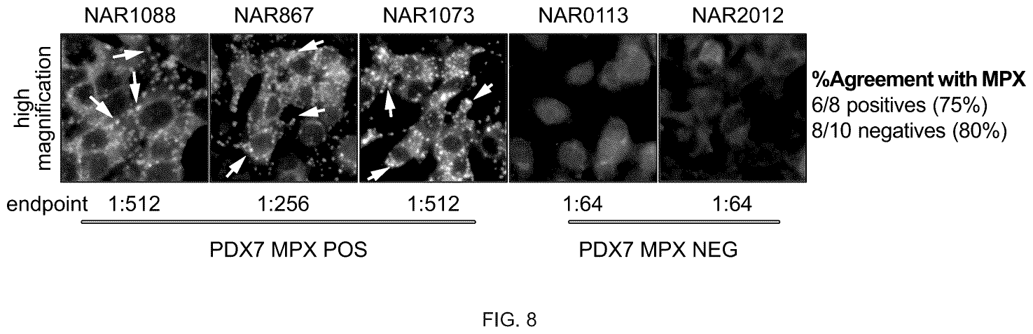

PDX7 peptides were conjugated to amino beads and screened against 574 samples from the United States using the direct assay format in which biotinylated peptide is used as a detector polypeptide. Using this approach, 8 positive samples were identified out of 574 tested (1.4% positivity; 98.6% negativity). The 8 positives were further evaluated on an IDEXX RMSF IFA for confirmation of positivity. 6 of the 8 PDX7 positive samples by multiplex were confirmed positive by IFA, suggesting strong sensitivity of PDX7 peptide for capturing anti-Rickettsia rickettsii antibodies. See FIG. 8 showing 75% agreement with IFA for positive samples (6/8 positives) and 80% agreement with negative samples (8/10 positives).

PDX7, TDX1779 and TDX1780 coated beads were incubated against RMSF negative (neg) or positive (pos) serum from the IDEXX Reference lab in Sacramento and detected using biotinylated TDX1779 as the conjugate. This data suggests that presumed negative serum is negative with these markers and presumed positive serum reacts. It is worth noting that all presumed positives are not expected to react because the marker is predicted to have much higher specificity for R. rickettsii infection whereas IFA cannot distinguish between Rickettsia species. See Table 1.

TABLE 1

| | | | RMSF IFA | RMSF IFA |
|---|---|---|---|---|
| | RMSF IFA | RMSF IFA | negative | positive |
| Analyte | Neg. reactivity | Pos. reactivity | agreement | agreement |
| PDX7 | 0/59 | 2/36 | 100% | 5.6% |
| TDX1779 | 2/59 | 6/36 | 96.6% | 16.7% |
| TDX1780 | 2/59 | 4/36 | 96/6% | 11.1% |

PDX6, PDX7, TDX1779 and TDX1780 (SEQ ID NOs: 1-4, respectively) were tested for their ability to specifically bind antibodies against R. rickettsii. Beads coated with PDX6, PDX7, TDX1779 or TDX1780 (SEQ ID NOs: 1-4, respectively) were incubated with each of 275 canine sera and detected using biotinylated TDX1779 (SEQ ID NO:3) as the conjugate in the direct format, using streptavidin-phycoerythrin as the label. The 275 sera were selected based on their test results for other tickborne pathogens (43 sera had each tested positive for antibodies against one of several tickborne pathogens as follows: 18 sera had tested positive for antibodies against Anaplasma ssp.; 11 sera had tested positive for antibodies against Ehrlichia canis or Ehrlichia ewingii; 14 sera had tested positive for antibodies against Borrelia burgdorferi) as well as their geographical distribution across the U.S. None of the 43 positive sera yielded a positive result with TDX1779 and TDX1780 (100% specificity) (SEQ ID NOs: 3-4, respectively). One of the 43 positive sera (i.e., one of the sera positive for antibodies against Anaplasma ssp.) yielded a positive result with PDX6 and PDX7 (at least 97.7% specificity) (SEQ ID NOs: 1-2, respectively). This data demonstrates that PDX6, PDX7, TDX1779 and TDX1780 (SEQ ID NOs: 1-4, respectively) each bind patient antibodies against R. rickettsii with high specificity. This data also demonstrates that a highly specific assay for antibodies against R. rickettsii can be built with PDX6, PDX7, TDX1779 and TDX1780 (SEQ ID NOs: 1-4, respectively).

### Example 2

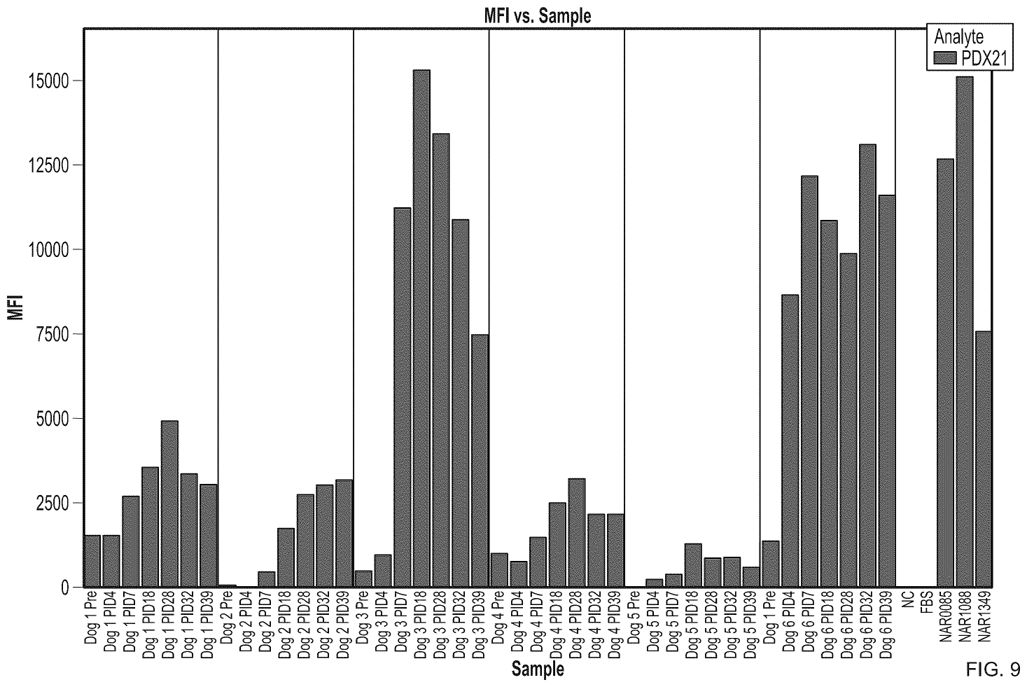

Serum from R. rickettsii experimentally infected canines was evaluated for reactivity to PDX21 polypeptide (SEQ ID NO:5) on the ABC multiplex platform using biotinylated rabbit anti-canine as the detector. In FIG. 9 individual dogs 1-6 (left to right) are separated by vertical black lines. For each experimentally infected canine, pre immune sera “Pre” was compared to sera from post-infection day 4, 7,18, 28, 32 and 39 as indicated by the numbers after the dogs. While each dog exhibited varying baseline reactivity, all six dogs showed increased reactivity to PDX21 from between days 7-18 post infection compared to respective pre-immune sera. Reactivity to PDX21 peaked by day 28-32 post infection and either plateaued or began declining thereafter. To the far right of FIG. 9 negative and positive controls are shown. NC=BSA/PBS as the sample, FBS=fetal bovine serum as the sample, positive controls (PC) are NAR0085, 1088 and 1349 are confirmed RMSF positive samples and showed strong reactivity to PDX21, highlighting the reactivity of VUT protein-derived PDX21 peptide to serum from R. rickettsii naturally and experimentally infected canine serum

We claim:

1. A method for treating a disease caused by Rickettsia rickettsii in a subject comprising administering a treatment if elevated levels of anti-Rickettsia rickettsii antibodies or specific binding fragments thereof that specifically bind to one or more polypeptides as set forth in SEQ ID NO:1, 2, 3, 4, 5, 6, or 7, a polypeptide comprising of amino acids 2-48 of SEQ ID NO: 1, or a polypeptide comprising amino acids 2-38 of SEQ ID NO:2 are present in a biological sample from the subject as compared to a control, wherein the treatment is administration of antibiotics, nucleic acid based drugs, anti-inflammatory drugs, immunomodulatory compounds, dietary supplements of copper (I) complex with glycine, pyruvate, and/or succinic acid, isothiocyanate functional surfactants, isooxazoline compositions, pyrazolo[1,5-a][1,3,5]triazine derivatives and pharmaceutically acceptable salts thereof, electrolytic acid water comprising free chlorine, proteasome inhibitors, polymorphs of the hydrochloride salt(S)-3-aminomethyl-7-(3-hydroxy-propoxy)-3H-benzo[c][1,2]oxaborol-1-ol, pyridinylamines, heterobicyclic compounds and pharmaceutically acceptable salts thereof, pyrazine derivatives, aromatic amidine inhibitors of trypsin-like proteases, or ATP synthase inhibitors.

2. The method of claim 1, wherein a quantity of anti-Rickettsia rickettsii antibodies or specific binding fragments thereof is determined via an immunoassay.

3. The method of claim 2, wherein one or more polypeptides as set forth in SEQ ID NO:1, 2, 3, 4, 5, 6, or 7, a polypeptide comprising of amino acids 2-48 of SEQ ID NO:1, or a polypeptide comprising amino acids 2-38 of SEQ ID NO:2 are immobilized to a support.

4. The method of claim 2, wherein complexes of the anti-Rickettsia rickettsii antibodies or specific binding fragments thereof and the one or more polypeptides as set forth in SEQ ID NO: 1, 2, 3, 4, 5, 6, or 7, a polypeptide comprising of amino acids 2-48 of SEQ ID NO:1, or a polypeptide comprising amino acids 2-38 of SEQ ID NO:2 are detected using one or more secondary antibodies or specific binding fragments thereof that specifically bind anti-Rickettsia rickettsii antibodies or specific binding fragments thereof.

5. The method of claim 4, wherein the secondary antibodies or specific binding fragments thereof comprise one or more tags or labels.

6. The method of claim 4, wherein the complexes are detected using one or more detector polypeptides comprising 90% or more sequence identity to a polypeptide as set forth in SEQ ID NO:1, 2, 3, 4, 5, 6, or 7, a polypeptide comprising of amino acids 2-48 of SEQ ID NO: 1, or a polypeptide comprising amino acids 2-38 of SEQ ID NO:2.

7. The method of claim 6, wherein the one or more detector polypeptides comprise a label or tag.

8. The method of claim 1, wherein the subject has been infected with Rickettsia for less than 7 days.

9. The method of claim 4, further comprising comparing an amount of the complexes in the sample to a control sample or control standard, wherein elevated levels of the complexes as compared to the control sample or control standard is an indication of a disease caused by Rickettsia rickettsii.

10. The method of claim 1, wherein the subject is a non-human animal.

11. The method of claim 1, wherein the subject is a human.

12. The method of claim 1, wherein the biological sample is blood, plasma, serum, or lymph fluid.

13. A fusion protein comprising two, three, four, five, six, seven or more polypeptides having 90% or more sequence identity to:
amino acids 2-48 of SEQ ID NO:1; or
amino acids 2-38 of SEQ ID NO:2.

14. The polypeptide of claim 13, wherein the fusion protein is lyophilized, desiccated, or dried.

15. The polypeptide of claim 13, wherein the fusion protein further comprises one or more labels or tags.

16. The polypeptide of claim 13, wherein the fusion protein is immobilized to a support.

17. A polypeptide consisting of:
amino acids 2-48 of SEQ ID NO:1 or amino acids 2-38 of SEQ ID NO:2, and
one or more secretory signal sequences, one or more epitope tags, or one or more secretory signal sequences and one or more epitope tags.

18. The polypeptide of claim 17, wherein the fusion protein is lyophilized, desiccated, or dried.

19. A kit for diagnosing a disease caused by Rickettsia rickettsii, the kit comprising:
(a) fusion protein comprising two, three, four, five, six, seven or more polypeptides having 90% or more sequence identity to:
amino acids 2-48 of SEQ ID NO:1; or
amino acids 2-38 of SEQ ID NO:2;
or
(b) a polypeptide consisting of:
amino acids 2-48 of SEQ ID NO:1 or amino acids 2-38 of SEQ ID NO:2, and
one or more secretory signal sequences, one or more epitope tags, or one or more secretory signal sequences and one or more epitope tag; and
(b) one or more reagents that facilitate binding of the one or more polypeptides to anti-Rickettsia rickettsii antibodies or specific binding fragments thereof present in a test sample.


SEQUENCE LISTING (23 sequences)

SEQ ID NO: 1 - protein, 48 aa, synthetic construct
  REGION 1..48: note = PDX6
  VARIANT 9: note = X - The X at position 9 can be E or A
  VARIANT 34: note = X - The X at position 34 can be G or S
CDYKKSLLXLRSSDEDDQGYATGYTTDEEELEEXNSTTGEELKKDISD

SEQ ID NO: 2 - protein, 38 aa, synthetic construct
  REGION 1..38: note = PDX7
  VARIANT 24: note = X - The X at position 24 can be G or S
CSSDEDDQGYATGYTTDEEELEEXNSTTGEELKKDISD

SEQ ID NO: 3 - protein, 172 aa, synthetic construct
  REGION 1..172: note = TDX1779
MDWTWRVFFLLALATGVHSCDYKKSLLELRSSDEDDQGYATGYTTDEEELEESNSTTGEELKKDISDDYKKSLLELRSSDEDDQGYATGYTTDEEELEESNSTTGEELKKDISDDYKKSLLELRSSDEDDQGYATGYTTDEEELEESNSTTGEELKKDISDAAAHHHHHHHH

SEQ ID NO: 4 - protein, 266 aa, synthetic construct
  REGION 1..266: note = TDX1780
MDWTWRVFFLLALATGVHSCDYKKSLLELRSSDEDDQGYATGYTTDEEELEESNSTTGEELKKDISDDYKKSLLELRSSDEDDQGYATGYTTDEEELEESNSTTGEELKKDISDDYKKSLLELRSSDEDDQGYATGYTTDEEELEESNSTTGEELKKDISDDYKKSLLELRSSDEDDQGYATGYTTDEEELEESNSTTGEELKKDISDDYKKSLLELRSSDEDDQGYATGYTTDEEELEESNSTTGEELKKDISDAAAHHHHHHHH

SEQ ID NO: 5 - protein, 38 aa, synthetic construct
  REGION 1..38: note = PDX21
MLFDKIKSKRKGKSMSNINAKFYIPLVSLLGVFIYLLN

SEQ ID NO: 6 - protein, 37 aa, synthetic construct
  REGION 1..37: note = PDX39
CKEVLYKSAYSLTVYISIFLVQKVYGNNGSVRALKNF

SEQ ID NO: 7 - protein, 38 aa, synthetic construct
  REGION 1..38: note = PDX40
CLFDKIKSKRKGKSMSNINAKFYIPLVSLLGVFIYLLN

SEQ ID NO: 8 - protein, 200 aa, synthetic construct
  REGION 1..200: note = The Rickettsia vitamin uptake transporter VUT family protein
MLFDKIKSKRKGKCMSNINAKFYIPLVSLLGVFIYLLNCFNKISQCSLVFVFLAITTNIISELYGRKRALIAVALCIIVSFGLLWNFNYYIHGRVIKGVVFASFVSVLLSTYCSTSIFSQLKPRCSLNTRNFASLIMCAVVDGIVMSGFFVNVFSTSKVLSIFYKEVLYKCAYSLTVYICIFLVQKVYGNNGSVRALKNF

SEQ ID NO: 9 - protein, 10 aa, synthetic construct
  REGION 1..10: note = HA epitope tag
YPYDVPDYAC

SEQ ID NO: 10 - protein, 11 aa, synthetic construct
  REGION 1..11: note = myc epitope tag
EQKLISEEDLC

SEQ ID NO: 11 - protein, 14 aa, synthetic construct
  REGION 1..14: note = V5 epitope tag
GKPIPNPLLGLDST

SEQ ID NO: 12 - protein, 13 aa, synthetic construct
  REGION 1..13: note = E-tag epitope tag
GAPVPYPDPLEPR

SEQ ID NO: 13 - protein, 11 aa, synthetic construct
  REGION 1..11: note = VSV-g epitope tag
YTDIEMNRLGK

SEQ ID NO: 14 - protein, 7 aa, synthetic construct
  REGION 1..7: note = 6xHis epitope tag
HHHHHHH

SEQ ID NO: 15 - protein, 12 aa, synthetic construct
  REGION 1..12: note = HSV epitope tag
QPELAPEDPEDC

SEQ ID NO: 16 - protein, 8 aa, synthetic construct
  REGION 1..8: note = FLAG epitope tag
DYKDDDDK

SEQ ID NO: 17 - protein, 73 aa, synthetic construct
  REGION 1..73: note = SCA2 auto chaperone region from Rickettsia rickettsii species
TALSPRLLSSNDSKNDKSSDYKKSLLELRSSDEDDQGYATGYTTDEEELEESNSTTGEELKKDISDYKKSLLA

SEQ ID NO: 18 - protein, 27 aa, synthetic construct
  REGION 1..27: note = SCA2 auto chaperone region from Rickettsia conorii, Rickettsia africae, Rickettsia japonica, and Rickettsia parkeri species
TALSPRLLSSNDSKNDKSSDDKKSLLA

SEQ ID NO: 19 - protein, 27 aa, synthetic construct
  REGION 1..27: note = SCA2 auto chaperone region from Rickettsia philipii species
TALSPRLLSSNDSKNDKSSDYKKSLLA

SEQ ID NO: 20 - protein, 27 aa, synthetic construct
  REGION 1..27: note = SCA2 auto chaperone region from Rickettsia massiliae species
TVLSPRLLCSNDSKNDKSSDDKKSLLA

SEQ ID NO: 21 - protein, 27 aa, synthetic construct
  REGION 1..27: note = SCA2 auto chaperone region from Rickettsia felis species
TALSPRLLSSNDSKNDKSSDDKKSLLV

SEQ ID NO: 22 - protein, 17 aa, synthetic construct
  REGION 1..17: note = SCA2 auto chaperone region from Rickettsia typhi species
NCLDTEEEVVHKEKIAD

SEQ ID NO: 23 - protein, 19 aa, synthetic construct
  REGION 1..19: note = SCA2 auto chaperone region from Rickettsia bellii species
IRAHPDLFPTVFNDPDTLA